(19) United States
(12) Patent Application Publication
(10) Pub. No.: US 2023/0304984 A1
(43) Pub. Date: Sep. 28, 2023

(54) A METHOD FOR THE EARLY DETECTION OF POTATO BACTERIAL DISEASES BASED ON CHARACTERIZING VOLATILE SIGNATURES

(71) Applicant: THE STATE OF ISRAEL, MINISTRY OF AGRICULTURE & RURAL DEVELOPMENT, AGRICULTURAL RESEARCH ORGANIZATION, Rishon Lezion (IL)

(72) Inventors: Zipora TIETEL, Givot Bar (IL); Leah TSROR, Beer Sheva (IL)

(21) Appl. No.: 18/020,518

(22) PCT Filed: Aug. 9, 2021
(86) PCT No.: PCT/IL2021/050969

§ 371 Date: Feb. 9, 2023

Related U.S. Application Data
(60) Provisional application No. 63/063,436, filed on Aug. 10, 2020.

(51) Int. Cl. G01N 33/00

(52) U.S. Cl. CPC G01N 33/0098

(57) ABSTRACT

The present application discloses a method for the early detection of bacterial diseases of potato tubers. More particularly, the present application discloses a method for identifying bacteria which cause blackleg and tuber soft rot diseases in potato tubers based on the identification of unique patterns of volatile compounds. The method disclosed herein can serve for the differentiation of non-inoculated samples from inoculated samples, distinguishing and identifying inoculation by different bacterial genera (Pectobacterium and Dickeya spp), different bacterial species and different bacterial sub-species. The detection relies on specific combinations of volatile compounds associated with each of the above-mentioned groups, which can serve as an applicable tool to detect tuber pathologies in storage rooms and shipments.

### FIELD OF THE INVENTION

The present disclosure relates to the early detection of Pectobacterium and Dickeya spp in potato tubers based on the identification of unique patterns of volatile compounds. More particularly, the detection distinguishes inoculated samples from non-inoculated samples, and inoculation by different bacterial genera (Dickeya from Pectobacterium), different bacterial species and different bacterial sub-species. The detection relies on specific combinations of volatile compounds associated with each group, which can serve as an applicable tool to detect tuber pathologies in storage rooms and shipments.

### BACKGROUND OF THE INVENTION

Potato blackleg and tuber soft rot diseases, caused by the pectinolytic bacteria Pectobacterium and Dickeya spp., result in severe yield losses worldwide. Early detection of pectinolytic bacterial infections is an important tool in disease management for sustainable potato production. The main goal of the current application is the development of a reliable and highly sensitive method for the detection and characterization of volatiles released by potato tubers infected with the major pectinolytic bacteria associated with potato soft rot.

Potato (Solanum tuberosum) is the fourth most important food crop and an integral part of the world's food supply. Potato blackleg and tuber soft rot diseases, caused by various species of Pectobacterium and Dickeya, result in severe yield losses worldwide and are a serious challenge to potato production in both the field and postharvest storage. These diseases are of a great concern due to climatic conditions during the growing season that favor disease expression and may result in the establishment of the pathogens in potato fields and their spread to weeds and other crops.

Different detection methods are available for determining the presence of pathogens in tubers, including microbiological, serological, immunological and molecular-based methods (see “Detection, identification and differentiation of Pectobacterium and Dickeya species causing potato blackleg and tuber soft rot: a review”. Czajkowski, R. et al, Annals of Applied Biology 166, 18-38, 2015). In addition, several reported methods are based on e-nose sensors (see “Detection of potato brown rot and ring rot by electronic nose: From laboratory to real scale”. Biondi, E., et al, Talanta 129, 422-430, 2014) or IR spectroscopy (see “Differentiation of Pectobacterium and Dickeya spp. phytopathogens using infrared spectroscopy and machine learning analysis.” Abu-Aqil, G. et al., Journal of Biophotonics, e201960156, 2020), using spectral signatures of plant pathogens. However, many of these approaches are destructive, time consuming and laborious, or lack the required sensitivity to detect infection at its very early stages. Specifically, latent infections by tuber-borne pathogens at an early stage of growth have no visible external symptoms, making their detection and control at this stage an even more challenging task. Thus, rapid, low cost and accurate detection of Pectobacterium and Dickeya at the species level is one of the most important steps towards disease management and prevention of yield losses.

Aroma-sensing has been suggested as a sensitive and accurate tool for early detection of pre-mature, non-visible infections in potato tubers during storage, both for seed and ware potato. Previous studies have shown that potato pathogens cause pathogen-specific alternations in the volatile profile of the contaminated tuber. Characteristic changes in the levels of specific volatile compounds upon pathogen inoculation of potato tubers, e.g. by P. carotovorum (previously named Erwinia carotovora), Pythium ultimum and Fusarium sambucinum were previously reported. Additional studies also demonstrated the high specificity of this method, enabling discrimination between Erwinia carotovorum subp. carotovorum, P. c. subsp. atrosepticum and F. sambicinum, as well as between Phytophthora infestans, P. ultimum, Botrytis cinerea compared with the non-inoculated sample. Subsequently, the use of a coupled sensor or an e-nose system was suggested for volatile sampling (see “Detection of potato brown rot and ring rot by electronic nose: From laboratory to real scale”. Biondi, E., et al, Talanta 129, 422-430, 2014, and “The development of a sensor system for the early detection of soft rot in stored potato tubers”. De Lacy Costello, B. et al, Measurement Science and Technology 11, 1685, 2000). Use of aroma profiling for detection of fungal and bacterial spoilage was described for other products as well, e.g. desserts and grains (see “Volatiles as an indicator of fungal activity and differentiation between species, and the potential use of electronic nose technology for early detection of grain spoilage.” Magan, N., Evans, P., Journal of Stored Products Research 36, 319-340, 2000, and “Identification of the bacteria and their metabolic activities associated with the microbial spoilage of custard cream desserts.” Techer, C. et al, Food microbiology 86, 103317, 2020).

GB patent application 2562275A to The University of Warwick discloses a system used for gas and/or volatile organic compound monitoring of an agricultural product in a storeroom, transportation container or bed or field. The system contains a set of gas sensor units. The gas sensors may detect gases, vapors and/or volatile organic compounds which may be indicative of a disease, such as bacterial soft rot, or a disorder such as black heart in the agricultural product or to monitor the condition of the agricultural product, such as freshness or ripeness, or track the level of a chemical used to treat the agricultural product, such as a sprouting suppressant.

In view of the prior art and given the various challenges related to monitoring plant diseases, there is still an unmet long-felt need for a rapid, sensitive and accurate method for the detection of bacterial crop diseases in their early stages and the identification of volatile biomarkers associated with the specific pathogens.

### BRIEF DESCRIPTION OF THE FIGURES

The accompanying figures, which are included to provide a further understanding of the invention and are incorporated in and constitute a part of this specification, illustrate embodiments of the invention and together with the description serve to explain the principles of the invention.

FIG. 1 depicting a discriminant analysis canonical plot showing the dichotomic distinction between Dickeya solani (Ds) and Dickeya dianthicola (Dd) strains;

FIG. 2 depicting a discriminant analysis canonical plot showing the dichotomic distinction between Pectobacterium strains: Pectobacterium carotovorum subsp. carotovorum (pcc) and Pectobacterium carotovorum subsp. brasiliense (pcbr);

FIG. 3 depicting a discriminant analysis canonical plot showing the dichotomic distinction between Pectobacterium carotovorum subsp. carotovorum (Pcc), carotovorum subsp. brasiliense (Pcbr), and Pectobacterium parmentieri (Pp);

FIG. 4A depicting the relative concentration of volatile compound (1-octanol) in all bacterial sub-species groups disclosed in the present application (Dd, Ds, Pcbr, Pcc, Pp); and

FIG. 4B depicting the relative concentration of a volatile compound (1-pentanol, 4-methyl) in all bacterial sub-species groups disclosed in the present application (Dd, Ds, Pcbr, Pcc, Pp).

### SUMMARY OF THE INVENTION

It is one object of the present invention to disclose a method for detecting a plant disease at its early stages, comprising steps of:
a. obtaining a plant sample;
b. extracting volatile compounds from said plant sample;
c. optionally, separating said volatile compounds by separation means;
d. optionally, identifying said volatile compounds; and
e. associating said volatile compounds with predetermined volatile reference patterns,

wherein, said detecting is executed on a group selected from: inoculated samples compared to non-inoculated samples, different genera of microorganisms causing said plant disease, different species of microorganisms causing said plant disease, different sub-species of microorganisms causing said plant disease, and any combination thereof.

It is another object of the present invention to disclose the method as described above, wherein said plant disease is potato blackleg, soft rot diseases and any combination thereof.

It is another object of the present invention to disclose the method as described above, wherein said plant is potato.

It is another object of the present invention to disclose the method as described above, wherein said plant sample is selected from a group consisting of whole plant, tuber, leaf, root, rhizome, flower, stem, cuttings, discs, macerated samples and any combination thereof.

It is another object of the present invention to disclose the method as described above, wherein said separation means are selected from a group consisting of chromatography and detection or identification means selected from a group consisting of mass spectrometry, spectrophotometry, NMR, near infra-red (NIR), flame ionization detector, nitrogen phosphorus detector, thermal conductivity detector, flame photometric detector, photoionization detector, electrolytic conductivity detector, quadrupole time of flight mass spectrometer, or their detection without separation or without identification by a sensor, a biosensor or an electronic nose or NIR detector and any combination thereof.

It is another object of the present invention to disclose the method as described above, wherein said predetermined volatile reference patterns are generated from plants of the same species as said plant sample, inoculated with microorganisms, analyzed for volatile compounds by said separation and identification means or other means known in the art, or in any other means, and saved as a reference to be compared against said plant sample.

It is another object of the present invention to disclose the method as described above, wherein said volatile compounds are selected from any organic or inorganic chemical produced by said plant.

It is another object of the present invention to disclose the method as described above, wherein said volatile compounds are selected from a group consisting of isoprenoids, terpenoids, alcohols, esters, ketones, alkanes, phenylpropanoids, benzenoids, fatty acids and their derivatives, amino acids derivatives, oxygenated volatile compounds, sulfur-containing compounds, and derivatives thereof and any combination thereof.

It is another object of the present invention to disclose the method as described above, wherein said predetermined volatile reference patterns used to discriminate between said inoculated sample compared to non-inoculated sample are selected from a group comprising: 3,5-Octadien-2-one, E,E-; Carveol; Acetic acid, methyl ester; Benzene, 1,2-dimethoxy; and Dimethyl ether, Valencene; Isopropyl Alcohol; 1,6-Dioxaspiro4.4nonane, 2-ethyl-; Acetoin (3-hydroxy-2-butanone); 2,3-Butanediol; and Octanoic acid ethyl ester and any combination thereof.

It is another object of the present invention to disclose the method as described above, wherein said predetermined volatile reference patterns used to discriminate between said genera of said microorganisms are selected from a group comprising: Hexanal; and 2,4-Heptadienal, E,E, Phenol, 2-methoxy; 2,3-Butanediol, R,R-; 2,4-Nonadienal, E,E-; UK1; 1-Hexanol; Phenol, 4-ethyl-2-methoxy-; and 3-Hepten-1-ol and any combination thereof.

It is another object of the present invention to disclose the method as described above, wherein said predetermined volatile reference patterns used to discriminate between said species of said microorganisms are selected from a group comprising: Thiophene, 2-pentyl, 1-Pentanol, 3-methyl-; 1-Butanol, 3-methyl-, acetate; 1-Butanol, 2-methyl; Valencene, 2-Octen-1-ol, E-; Phenol, 2-methoxy-; Carveol; 1-Butanol, 2-methyl; 1-Hexanol, 1-Pentanol, 3-methyl; 1-Octanol; 2,3-Pentanedione; Cyclopentane, methyl-; 1-Pentanol, 4-methyl-; Phenylethyl Alcohol and any combination thereof.

It is another object of the present invention to disclose the method as described above, wherein said predetermined volatile reference patterns used to discriminate between said subspecies of said microorganisms are selected from a group comprising: 2,3-Octanedione; and 2-Hexenal, 2-methyl, Cyclopentane, methyl; 2-Octenal, E-; 3-Nonen-1-ol, Z-; 1-Octen-3-ol; and 2,4-Nonadienal, E,E-; 1-Butanol, 2-methyl-; 1-Propanol, 2-methyl-; 1-Penten-3-ol; 2-Decenal, Z-; Fenchol; 2-Undecanone; 2,3-Butanediol, R,R; 1-Pentanol, 2-methyl-; 2-Octenal, E-; Cyclopentane, methyl- and any combination thereof.

It is another object of the present invention to disclose the method as described above, wherein said microorganisms are selected from bacteria, fungi, viruses and any combination thereof. It is another object of the present invention to disclose the method as described above, wherein said bacteria are selected from a group consisting of: Pectobacterium and Dickeya and any combination thereof.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a method for the detection of blackleg and soft rot diseases in their early stages in plants based on specific volatile signatures. The disclosed method will allow early detection of contaminated batches and will avoid using contaminated vegetative propagative materials for planting, thus reduce soil damage and food losses.

As used herein after, the term “about” refers to any value being up to 25% lower or greater the defined measure.

As used herein after, the term “volatile/volatile compounds/volatile metabolites/volatile biomarkers” refers to naturally occurring organic chemicals with unique physical and chemical characteristics (such as specific boiling points, distinct odors or high vapor pressure at ordinary room temperature). Plants produce and emit an extraordinary number of volatile compounds as secondary metabolites, such as isoprenoids, alcohols, esters, ketones, alkanes terpenoids, oxygenated volatile compounds, phenylpropanoids, benzenoids, fatty acids and their derivatives, amino acid derivatives, alcohols, esters, ketones, alkanes and in some species sulfur-containing compounds and derivatives thereof.

As used herein after, the term “differentiating volatile biomarkers” refers to volatile metabolites which contributed to the optimized discriminant analyses presented in the present application. Those discriminant analyses were conducted in order to distinguish groups of inoculated plant and non-inoculated plants, groups of different bacterial genera (Dickeya from Pectobacterium), different bacterial species and different bacterial sub-species. In contrast, the term “supporting volatile biomarkers” as used herein refers to volatile metabolites which did not contribute to the generation of the discriminant analyses presented in the present application, even though they were found to be statistically significant and had high fold change values.

As used herein after, the term “blackleg” refers to a potato disease caused by pectolytic bacteria, mainly Pectobacterium and Dickeya spp. The disease is characterized among other things by stunting, wilting, chlorosis (yellowish foliage), and blackening and decay of the lower stem portion of the plant. This disease can spread from infected to healthy plants in the field via wind, aerosol or insects, and infect the tubers through open lenticels or wounds.

As used herein after, the term “soft rot” refers to various plant diseases caused by bacteria, most commonly gram-negative bacteria. This type of diseases is destructive, and it can affect almost all plant families. The bacteria can target numerous plant organs, such as storage organs and stems and use pectolytic enzymes to disintegrate and macerate the plant cells and to feed on its nutrients. As a result, the plant tissues become watery and soft. Soft rot diseases can be transmitted from infected plants to healthy plants during storage or shipments by physical interaction.

As used herein after, the term “seed tuber” refers to potato tubers which are used for propagation and multiplication of the crop. The tubers can be planted whole or after being cut into pieces.

The present invention provides a rapid, accurate and sensitive method for the detection of blackleg and soft rot diseases in early stages in potato tubers. The method is based on identifying specific patterns or signatures of volatile compounds produced in the potato tuber as a result of an infection and associating them with the presence of the pathogenic bacteria. The method can readily discriminate non-infected and infected samples, distinguish between bacterial genera (Pectobacterium and Dickeya) and between species and sub-species of these bacteria as well.

The current application discloses a reliable and highly sensitive method for the detection, characterization and relative quantification of the volatile compounds released by potato tubers infected with the pectinolytic bacteria Pectobacterium and Dickeya.

In a preferred embodiment of the present invention, the method is used to determine the unique volatile compounds developed by the potato tuber during the very early stages of the bacterial infection.

In yet another preferred embodiment of the present invention, the method is used to identify the specific volatile biomarkers associated with each type of bacteria (Pectobacterium and Dickeya).

In yet another preferred embodiment of the present invention, the method is used to identify specific volatile biomarkers of non-infected potato tubers compared to infected tubers, of Pectobacterium-infected tubers compared to Dickeya-infected tubers and tubers infected with their respective species and subspecies.

The current application and method thereof can be incorporated in other detection systems or be further developed to form unique and designated systems, to be utilized as biosensors or e-nose for potato diseases. The current application can serve for the detection of bacterial diseases in numerous settings, for example and in a non-binding manner, storage rooms, greenhouses and shipments.

Using volatile biomarkers for the early detection of microbial infection is advantageous over other methods in several aspects: (1) it is a very specific and sensitive approach; (2) this method is non-destructive, unlike other methods currently in use, allowing the analyzed tubers to be later consumed; (3) the most pronounced vantage of volatile sensing (by any form of sensing known in the art) is that the detection is performed in a storage room atmosphere, on bulk rather than a single tuber. Thus, a grower or pack house operator can save the trouble of searching the specific infected tuber in order to inspect it. In addition, continuous sampling of the atmosphere will notify the presence of any undesired biomarkers and allow proper handling of the situation accordingly. Furthermore, such monitoring can be carried out while shipping the tubers, indicating problematic shipments ahead of time, even before they have reached their destination.

Bacterial diseases caused by Dickeya and Pectobacterium species are among the most severe problems also in production of potato seed tubers. As infections are difficult to prevent when occurring, pre-expression identification is crucial, being the main step in managing these diseases. Potato tubers are used for multiplication, and as nutrimental source. Hence, the adequate treatment will differ accordingly. When an initial bacterial activity is detected among tubers that are designated as food source, batches with latent infections can be distributed earlier and save the entire stock. However, when latent infection is detected in seed tubers, it would be recommended to avoid planting. Therefore, it is of tremendous importance to detect diseases in seed tubers in their early stages, so only specific batches or shipments, suspected of being infected, can be properly managed and taken care of without having to destroy other batches.

To the inventors' knowledge, this is the first disclosure reporting in detail specific volatile biomarkers to indicate Pectobacterium and Dickeya infection in potato tubers, in addition to specifying differentiating volatile biomarkers for discrimination at sub-species levels. Sensor monitoring of storage rooms atmosphere and of tuber shipments is thus suggested as a powerful tool which would allow the early detection of the most common diseases, reducing significant losses and severe economic damages to growers and distributers, also reducing global food loss.

The various volatile patterns of infected potato tubers disclosed in the current application were obtained as follows:

Five bacterial strains from each of the following: Dickeya solani (Ds), P. carotovorum subsp. brasiliense (Pcbr), P. carotovorum subsp. carotovorum (Pcc), and four strains each of Pectobacterium parmentieri (Pp) and Dickeya dianthicola (Dd) were included in the current application (Table 1), all in triplicate.

All bacterial strains used in the current application were isolated from potato tubers or plants, or from wash water sampled during the process of packing ware potatoes in a commercial pack house.

TABLE 1
Details on the Dickeya and Pectobacterium species and strains included
in the current application.

| Genus | Species | Strain | Bacterial source |
|---|---|---|---|
| Pectobacterium | carotovorum | CFBP 6617 | Reference strain |
| | subsp. | | (Finland) |
| | brasiliense | 55 | Progeny tuber |
| | (Pcbr) | 97 | Plant |
| | | 133 | Seed tuber |
| | | | Netherlands |
| | | 185 | Wash water |
| | carotovorum | G414 | Progeny tuber |
| | subsp. | 6 | Progeny tuber |
| | carotovorum | 13 | Progeny tuber |
| | (Pcc) | 31 | Progeny tuber |
| | | 6/17 | Wash water |
| | parmentieri | 1958 | Reference strain |
| | (Pp) | | (Netherlands) |
| | | F1 | Wash water |
| | | F4 | Wash water |
| | | F5 | Wash water |
| Dickeya | solani | 4 | Plant |
| | (Ds) | 23 | Plant |
| | | 82 | Plant |
| | | 95 | Seed tuber |
| | | | (imported from |
| | | | Germany) |
| | | 102 | Seed tuber |
| | | | (imported from |
| | | | Germany) |
| | dianthicola | 74 | Seed tuber |
| | | | (imported from |
| | | | Netherlands) |
| | (Dd) | 114 | Plant |
| | | 246 | Wash water |
| | | 238 | Wash water |

Tubers or stems were washed, surface sterilized, macerated in sterile distilled water, and the suspensions were plated on a crystal violet pectate medium (CVP). Wash water samples (200 mL) were filtered through rough filter paper followed by fine Millipore (45 um) filter paper. The Millipore filter paper was re-suspended in 2 mL sterile distilled water, and suspensions were plated on CVP. Cavity forming bacteria were transferred to nutrient agar and grown at 33° C. for 24 h, to obtain pure colonies for further characterization. All strains were identified and characterized by several molecular methods, namely PCR and RT-PCR, and part of them were further characterized by dnaX or gapA sequencing. In addition, all strains were tested for their pectinolytic activity on potato tubers (cv. Vivaldi) using a tuber maceration assay. Bacterial suspensions for inoculation were prepared by culturing strains on nutrient agar (NA) at 27° C. for 48 h. A single colony was transferred to NB and grown at 27° C. (200 rpm) for 24 h after which optical density was adjusted to an absorbance of 0.2 at 600 nm using a Biomat3 spectrophotometer (Thermo Scientific). Bacterial suspensions were diluted and plated on NA to verify concentration. An optical density (OD) value of 0.2 was equivalent to ˜108 cfu mL⁻ 1. Tubers were washed in running tap water, surface-sterilized with hypochlorite for 10 min, and air-dried in a laminar chamber. Ten tubers per strain were pierced with a sterile micropipette tip (1000 μL), and ten μL of bacterial suspension was injected into the wound. Non-inoculated controls were injected with sterile distilled water. Tubers were placed in a moist chamber at 30° C. for 48 h. The tubers were then sliced at the inoculation point and macerated tissue was scraped off and weighed.

Bacterial suspensions and tubers for inoculation were prepared as described in the maceration assay. Potato plugs (1 cm diameter; 0.5 cm height) were cut into 10 mL glass vials, ten replicates for each strain. Ten μL of bacterial suspension was injected into the plug. The vials were incubated at 30° C. for 48 h.

Sample Preparation:

Aroma volatiles were extracted from the inoculated potato discs as follows: immediately after 48 h of incubation, 1 mL of 320 g L⁻¹-aqueous NaCl solution, containing 0.05 μL of 1-pentanol (5 μL L⁻¹) solution as internal standard, was added directly to the vial containing the inoculated potato disc. Vials were then stored in −20° C. pending analysis. Aroma volatiles were determined in three replicate measurements for each strain.

Solid Phase Microextraction (SPME):

Analysis of aroma volatiles was carried out using gas chromatography coupled with mass spectrometry (GC-MS). Prior to analysis, samples were allowed to equilibrate for 5 min at 40° C. and then incubated at the same temperature for an additional 30 min, with agitation at 250 rpm. Volatiles were extracted by solid-phase microextraction (SPME) using 1 cm long stable flex fibers coated with a 50/30 μm layer of divinylbenzene/carboxen/polydimethylsiloxane (DVB/CAR/PDMS) (Supelco, Bellefonte, PA, USA). The samples were introduced to the inlet using an auto-sampler PAL RSI 85.

Gas Chromatography coupled with Mass Spectrometer detector (GC-MS):

Following incubation, the fiber was desorbed for 2 min at 250° C. in the GC inlet, programmed to splitless mode. 7890B mode gas chromatograph (Agilent, Palo Alto, CA, USA) equipped with an HP-5 column (60 m×0.25 mm i.d.×0.25 μm film thickness) (J&W Scientific, Folsom, CA, USA). The oven was programmed to run at 50° C. for 2 min, then ramp up to 180° C. at 4° C. min⁻¹, isothermal hold at 180° C. for 2 min, then ramp up to 270° C. at 20° C. min⁻¹ and finally to remain at that temperature for 10 min. The helium carrier gas flow was set at 1 mL min⁻¹. The effluent was transferred to a Model 5977B MS detector (Agilent), transferline heated to 325° C. The MS was set to scan from m/z 40 to 350 at 7.72 scans s⁻¹ in positive ion mode, and mass spectra in electron impact mode were generated at 70 eV.

Identification and quantification of volatile compounds:

Chromatographic peaks were tentatively identified by comparing the mass spectrum of each component with the National Institute of Standards and Technology (NIST) 2006, 2014 and 2017 mass spectral library. Identification of aroma volatiles was further confirmed by calculating their linear retention indices (RIs) using a series of n-alkanes (C5-C20) and comparing their values with various published databases: Pherobase (www.pherobase.com), Flavornet (www.flavornet.org/flavornet.html), NIST Chemistry WebBook (webbook.nist.gov/chemistry/) and the LRI database (www.odour.org.uk/lriindex.html). We only considered volatiles that met the following criteria: (i) the compound was identified by MS with high probability (≥90%) and (ii) the calculated RI differed by less than ±15 from the estimated value available in public domain databases. For peak area normalization, the area of each compound was divided by the area of the internal standard added in the known amount specified herein.

Statistical Analysis:

Statistical analysis was carried out using the JMP statistical package and MetaboAnalyst 4.0. A minimum of three independent replicates were conducted for each of the five strains. Using Metaboanalyst, means of replicates were subjected to Tukey-Kramer honest significant difference (HSD; P≤0.05), and Fold Change (FC) data was extracted. In addition, biomarker analysis was performed. Based on these three analyses, an inclusive list was curated of metabolites which were either biomarkers, had high FC (FC>50 for increasing, or FC>2 for decreasing) or were statistically significant. Using the JMP statistical package, a group Linear Discriminant Analysis was carried out, in a stepwise manner, and caconical plots were optimized. The metabolites which contributed to the optimized discriminant analysis were defined as “differentiating biomarkers”, while those which were significant or had high FC but did not contribute to the discrimination were defined as “supportive biomarkers”.

### Example 1

The volatile fingerprints of potato discs inoculated with five bacterial species were profiled in order to identify specific biomarkers for each species. The first level of discrimination among samples was differentiating inoculated samples from the non-inoculated controls. Some compounds were present only in the inoculated samples, and vice versa for other compounds. Therefore, these volatiles were defined as inoculation-specific, and can be used as general inoculation indicators. Five compounds were defined as “differentiating biomarkers”, as their use for discriminant analysis was sufficient to receive an optimized discrimination between the two groups: 3,5-Octadien-2-one, E,E-; Carveol; Acetic acid, methyl ester; Benzene, 1,2-dimethoxy; and Dimethyl ether. Reference is now made to Table 2 presenting the fold change of these biomarkers, between inoculated and non-inoculated samples.

TABLE 2
Differentiating volatile biomarkers for discriminating between non-
inoculated and inoculated samples.

| Comparison | Differentiating volatile biomarkers | Fold Change | P |
|---|---|---|---|
| non- | 3,5-Octadien-2-one, E,E- | 6.59 | 0.047 |
| inoculated / | Carveol | 8.64 | 0.014 |
| Inoculated | Acetic acid, methyl ester | 0.007 | 0.002 |
| | Benzene, 1,2-dimethoxy- | 0.034 | P < 0.0001 |
| | Dimethyl ether | 0.003 | P < 0.0001 |

In addition, six other volatiles were supporting biomarkers (meaning they are not the main, most pronounced differentiating volatile biomarkers used to distinguish between the different groups, even though they are still statistically significant and have high fold change values): Valencene; Isopropyl Alcohol; 1,6-Dioxaspiro4.4nonane, 2-ethyl-; Acetoin (3-hydroxy-2-butanone); 2,3-Butanediol; and Octanoic acid ethyl ester (Table 3). Although some of these compounds were significant and showed high FC ratios, they did not considerably affect group discrimination. Nevertheless, they are included as additional biochemical information, which may shed some light on the pathways that are activated during inoculation.

TABLE 3
Supporting volatile biomarkers for discriminating between non-
inoculated and inoculated samples.

| | | Fold | |
|---|---|---|---|
| Comparison | Supporting biomarker | Change | P |
| Control / | Valencene | 0.008 | P < 0.0001 |
| Inoculated | Isopropyl Alcohol | 0.004 | P < 0.0001 |
| | 1,6-Dioxaspiro4.4nonane, | 0.001 | P < 0.0001 |
| | 2-ethyl- | | |
| | Acetoin | 0.001 | P < 0.0001 |
| | 2,3-Butanediol | 0.003 | P < 0.0001 |
| | Octanoic acid, ethyl ester | 0.012 | P < 0.0001 |
| | Phenol, 2-methoxy- | 0.177 | 0.015 |

### Example 2

The next level of discrimination was between genera, i.e. Dickeya (D) and Pectobacterium (P). Three volatiles were identified as differentiating biomarkers: unknown (UK) 2; Hexanal; and 2,4-Heptadienal, E,E (Table 4).

TABLE 4
Differentiating volatile biomarkers for discriminating between
bacterial genera (Dickeya [D] and Pectobacterium [P]).

| Comparison | Differentiating biomarkers | Fold Change | P |
|---|---|---|---|
| D / P | UK2 | 20.61 | 0.005 |
| | Hexanal | 5.13 | n.s |
| | 2,4-Heptadienal, E,E- | 5.85 | n.s |

The supporting volatile biomarkers for this classification included Phenol, 2-methoxy; 2,3-Butanediol, R,R-; 2,4-Nonadienal, E,E-; UK1; 1-Hexanol; Phenol, 4-ethyl-2-methoxy-; and 3-Hepten-1-ol (Table 5). Therefore, clustering of group D from P can be readily done based on the relative levels of these three volatile biomarkers, with very high prediction scores.

As for unknown (UK) compounds, retention times were 22.0899 and 28.7745 minutes and retention indices were 1085 and 1265 for UK1 and UK2, respectively.

TABLE 5
Supporting volatile biomarkers for discriminating between
bacterial genera (Dickeya [D] and Pectobacterium [P]).

| | | Fold | |
|---|---|---|---|
| Comparison | Supporting biomarkers | Change | P |
| D / P | 2,3-Butanediol, R,R | 0.204 | 0.0002 |
| | 2,4-Nonadienal, E,E- | 4.90 | 0.094 |
| | UK1 | 0.495 | n.s |
| | 1-Hexanol | 2.77 | 0.013 |
| | Phenol, 4-ethyl-2- | 0.084 | 0.043 |
| | methoxy- | | |
| | 3-Hepten-1-ol | 4.28 | 0.006 |

### Example 3

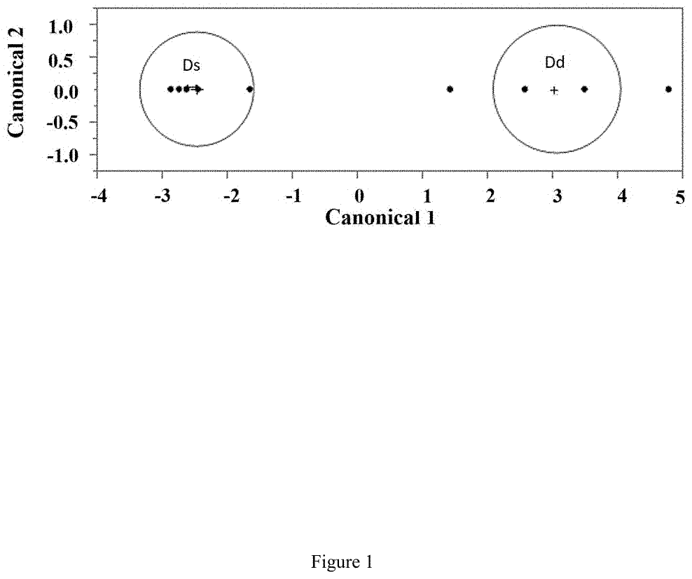

To classify between species, i.e. Dickeya solani (Ds) and Dickeya dianthicola (Dd), the relative concentrations of Thiophene, 2-pentyl-, was identified as a differentiating volatile biomarker (Table 6). The supporting volatile biomarkers included 1-Pentanol, 3-methyl-; 1-Butanol, 3-methyl-, acetate; 1-Butanol, 2-methyl; and Valencene (Table 7). Thus, Ds can be unequivocally discriminated from Dd (as illustrated in FIG. 1, depicting a discriminant analysis canonical plots showing the dichotomic distinction between the Dickeya species: Dickeya solani (Ds) and Dickeya dianthicola (Dd)). When sub-grouping Pectobacterium to species, i.e. Pectobacterium carotovorum (Pc) and Pectobacterium parmentieri (Pp), six differentiating volatile biomarkers were characterized: 2-Octen-1-ol, E-; Phenol, 2-methoxy-; Carveol; 1-Butanol, 2-methyl; UK2; and 1-Hexanol (Table 6). The supporting volatile biomarkers for Pc vs. Pp separation included 1-Pentanol, 3-methyl; 1-Octanol; 2,3-Pentanedione; Cyclopentane, methyl-; 1-Pentanol, 4-methyl-; and Phenylethyl Alcohol (Table 7).

TABLE 6
Differentiating volatile biomarkers for bacterial species discrimination.

| Comparison | Differentiating biomarkers | Fold Change | P |
|---|---|---|---|
| Dd / Ds | Thiophene, 2-pentyl- | 8.67 | 0.003 |
| Pc / Pp | 2-Octen-1-ol, E- | 0.059 | 0.004 |
| | Phenol, 2-methoxy- | 0.186 | 0.047 |
| | Carveol | 0.021 | 0.050 |
| | 1-Butanol, 2-methyl- | 20.86 | P < 0.0001 |
| | UK2 | 0.080 | 0.037 |
| | 1-Hexanol | 0.198 | 0.001 |

TABLE 7
Supporting volatile differentiating biomarkers
for bacterial species discrimination.

| | | Fold | |
|---|---|---|---|
| Comparison | Supporting biomarkers | Change | P |
| Dd / Ds | 1-Pentanol, 3-methyl- | 7.69 | 0.018 |
| | 1-Butanol, 3-methyl-, | 24.86 | n.s |
| | acetate | | |
| | 1-Butanol, 2-methyl- | 25.86 | n.s |
| | Valencene | 3.23 | 0.014 |
| Pc / Pp | 1-Pentanol, 3-methyl- | 4.58 | 0.0003 |
| | 1-Octanol | 0.25 | 0.0004 |
| | 2,3-Pentanedione | 7.16 | 0.050 |
| | Cyclopentane, methyl- | 19.05 | 0.0004 |
| | 1-Pentanol, 4-methyl- | 5.85 | 0.004 |
| | Phenylethyl Alcohol | 4.71 | 0.019 |
| | UK2 | 0.457 | 0.037 |

### Example 4

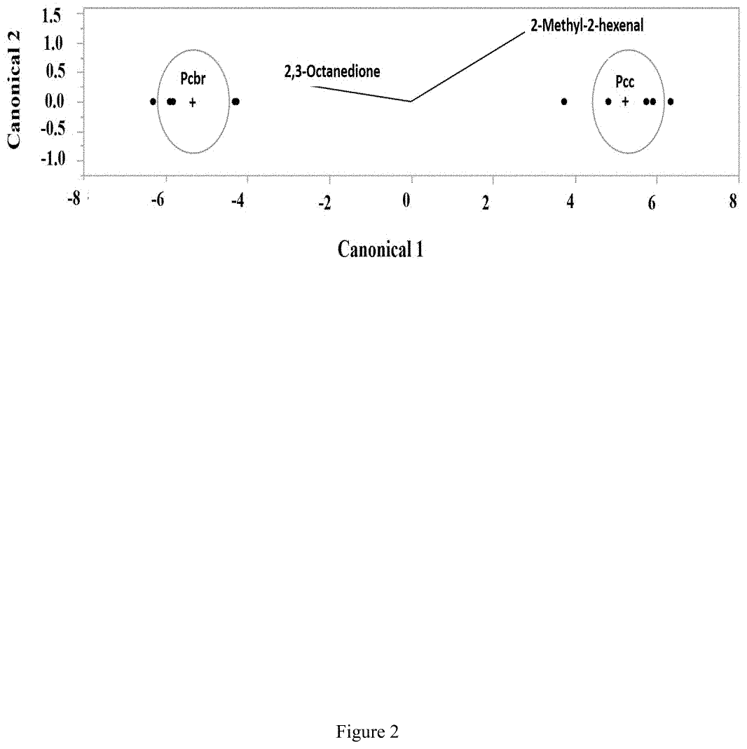

The finest level of separation was between sub-species, Pectobacterium carotovorum subsp. carotovorum (Pcc) and carotovorum subsp. Brasiliense (Pcbr), both belong to the same species (P. carotovorum). These sub-species can be further distinguished from one another based on the relative levels of two differentiating volatile biomarkers: 2,3-Octanedione; and 2-Hexenal, 2-methyl (Table 8). The supporting volatile biomarkers were UK2; Cyclopentane, methyl; 2-Octenal, E-; 3-Nonen-1-ol, Z-; 1-Octen-3-ol; and 2,4-Nonadienal, E,E- (Table 9). The differentiating volatile biomarkers allowed a good separation in discriminant analysis as depicted in FIG. 2. presenting the corresponding discriminant analysis canonical plots.

TABLE 8
Differentiating volatile biomarkers for
bacterial sub-species discrimination.

| Comparison | Differentiating biomarkers | Fold Change | P |
|---|---|---|---|
| | 2,3-Octanedione | 4.07 | 0.009 |
| Pcbr / Pcc | 2-Hexenal, 2-methyl- | 0.45 | 0.041 |

TABLE 9
Supporting volatile biomarkers for bacterial sub-species discrimination.

| | | Fold | |
|---|---|---|---|
| Comparison | Supporting biomarker | Change | P |
| Pcbr / Pcc | Cyclopentane, methyl- | 2.44 | 0.0004 |
| | 2-Octenal, E- | 0.3343 | 0.029 |
| | 3-Nonen-1-ol, Z- | 0.3561 | 0.0001 |
| | 1-Octen-3-ol | 2.29 | 0.044 |
| | 2,4-Nonadienal, E,E- | 0.359 | 0.034 |

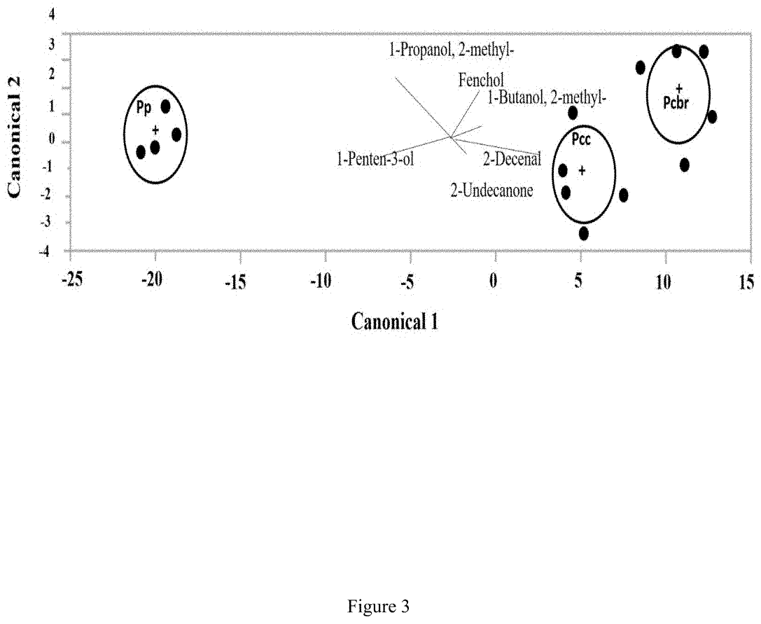

Furthermore, all three P groups were distinguished from one another as can be shown in FIG. 3, depicting the corresponding discriminant analysis canonical plots. The six differentiating volatile biomarkers used for the analysis were 1-Butanol, 2-methyl-; 1-Propanol, 2-methyl-; 1-Penten-3-ol; 2-Decenal, Z-; Fenchol; and 2-Undecanone (Table 10).

TABLE 10
Differentiating volatile biomarkers for Pectobacterium
sub-species discrimination.

| Comparison | Differentiating biomarkers | P |
|---|---|---|
| Pp, Pcc, Pcbr | 1-Butanol, 2-methyl- | P < 0.0001 |
| | 1-Propanol, 2-methyl- | 0.0003 |
| | 1-Penten-3-ol | 0.0001 |
| | 2-Decenal, Z- | 0.039 |
| | Fenchol | 0.002 |
| | 2-Undecanone | 0.007 |

The supporting volatile biomarkers included 2,3-Butanediol, R,R; 1-Pentanol, 2-methyl-; 2-Octenal, E-; and Cyclopentane, methyl-(Table 11).

TABLE 11
Supporting volatile biomarkers for Pectobacterium
sub-species discrimination.

| Comparison | Supporting biomarker | P |
|---|---|---|
| Pp, Pcc, Pcbr | 2,3-Butanediol, R,R | 0.001 |
| | 1-Pentanol, 2-methyl- | 0.053 |
| | 2-Octenal, E- | 0.045 |
| | Cyclopentane, methyl- | 0.0002 |

In order to identify biomarkers that enable segregation between all five sub-species groups (of both Pectobacterium and Dickeya), biomarker analysis was performed as well. The eight differentiating volatile biomarkers included 2,3Butanediol, R,R-; Thiophene, 2-pentyl-; Dimethyl ether; Cyclopentane, methyl-; 3-Nonen-1-ol, Z-; 3-Octanone; 2-Pentanone; and 1-Hexanol (Table 12). The supporting volatile biomarkers included 1-Pentanol, 3-methyl-; 1-Pentanol, 4-methyl; 1-Penten-3-ol; 1-Octanol; 1-Butanol, 2-methyl; and Fenchol (Table 13). Using these eight differentiating biomarkers, a separation between all five groups was successfully achieved.

TABLE 12
Differentiating volatile biomarkers in discriminant
analysis of Pectobacterium and Dickeya sub-species.

| Comparison | Differentiating biomarkers | P |
|---|---|---|
| Dd, Ds, Pp, | 2,3 Butanediol, R,R- | P < 0.0001 |
| Pcc, Pcbr | Thiophene, 2-pentyl- | 0.0001 |
| | Dimethyl ether | P < 0.0001 |
| | Cyclopentane, methyl- | 0.0001 |
| | 3-Nonen-1-ol, Z- | 0.0001 |
| | 3-Octanone | P < 0.0001 |
| | 2-Pentanone | 0.001 |
| | 1-Hexanol | 0.005 |

TABLE 13
Supporting volatile biomarkers in discriminant analysis of
Pectobacterium and Dickeya sub-species.

| | Supporting | |
|---|---|---|
| Comparison | biomarkers | P |
| Ds, Dd, Pp, Pcc, | 1-Pentanol, 3-methyl- | 0.001 |
| Pcbr | 1-Pentanol, 4-methyl- | 0.010 |
| | 1-Penten-3-ol | 0.014 |
| | 1-Octanol | 0.015 |
| | 1-Butanol, 2-methyl- | 0.001 |
| | Fenchol | P < 0.0001 |

The results of the current application show that the disclosed method is very versatile, and can indicate a wide range of situations, from whether infection has occurred, to the presence of specific subspecies. Based on volatile profiles and specific biomarkers, it was made possible to discriminate samples at various levels:
(1) inoculated vs. non-inoculated [Tables 2-3];
(2) genus level, Pectobacterium vs. Dickeya [Tables 4-5];
(3) species level, Ds vs. Dd, and Pc vs. Pp [Tables 6-7];
(4) sub-species level, Pcc vs. Pcbr [Tables 8-11].

Separation of Pectobacterium into three groups, namely Pp, Pcc and Pcbr was also possible, in addition to the separation of all five studied sub-species groups [Tables 12-13].

It is worth mentioning that the number of differentiating biomarkers was surprisingly small, usually varying from one to six, which emphasizes the specificity of the method. According to the results of the current application, with only as few as 25 volatile compounds acting as differentiating biomarkers, all five sub-species could be detected and discriminated from one another.

### Example 5

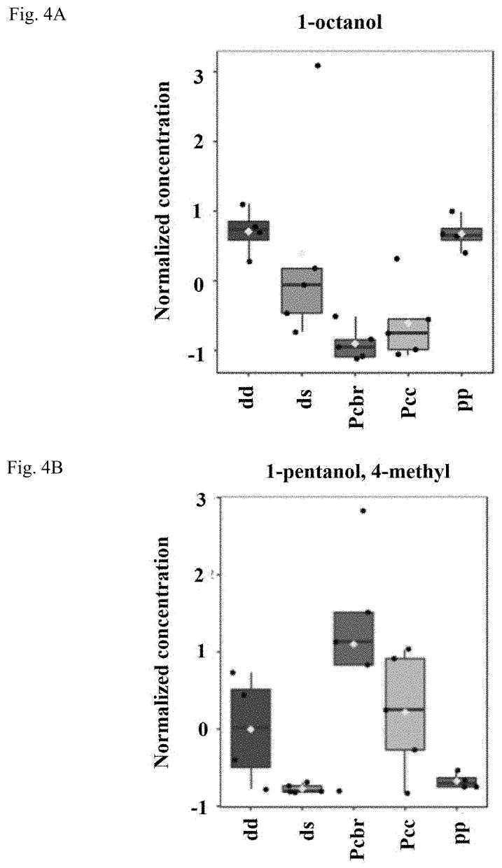

Although groups were distinguished in discriminant analysis in all cases based on characteristic volatiles, in some cases the separation was sub-optimal. However, for detection purposes the suggested differentiating biomarkers can be used separately or combined, thus increasing method specificity. For example, 1-octanol presence may indicate an inoculation by either Dd (Dickeya dianthicola) or Pp (Pectobacterium parmentieri). Reference is now made to FIG. 4A, graphically showing the relative (normalized) concentration of 1-octanol in all five bacterial sub-species groups (dd, ds, Pcbr, Pcc, pp).

Adding a biomarker which can distinguish between P and D groups will supply a definitive answer regarding the nature of the inoculation. In the case of 1-Pentanol, 4-methyl (as illustrated in FIG. 4B), a two-step validation of D vs. P followed by Pcc (Pectobacterium carotovorum subsp. Carotovorum) vs. Pcbr (carotovorum subsp. brasiliense) would be informative in specifically indicating the origin of inoculation.

The results of the current application are significant from a practical and industrial points of view, as the detection of the differentiating biomarkers, with or without the supporting biomarkers, could be further coupled with a sensor, and utilized for monitoring inoculation during early stages inside pack houses or shipments.

In addition, the current application discloses a list of volatile compounds identified for the first time as being produced in potato tubers as a result of bacterial infections (caused by Pectobacterium and Dickeya). These volatile compounds include in a non-binding example: 3,5-Octadien-2-one, E,E-; Carveol; Benzene, 1,2-dimethoxy-; 2,4-Heptadienal, E,E; Thiophene, 2-pentyl-; 2-Octen-1-ol, E-; Phenol, 2-methoxy-; 2,3-Octanedione; 2-Hexenal, 2-methyl-; 1-Penten-3-ol; 2-Decenal, Z-; Fenchol; 2-Undecanone; Cyclopentane, methyl-; 3-Nonen-1-ol, Z-; 3-Octanone; Valencene; 1,6-Dioxaspiro4.4nonane, 2-ethyl-; Octanoic acid ethyl ester; 2,4-Nonadienal, E,E; Phenol, 4-ethyl-2-methoxy-; 3-Hepten-1-ol; 1-Pentanol, 3-methyl-; 2,3-Pentanedione; 1-Pentanol, 4-methyl-; 1-Octen-3-ol; and 1-Pentanol, 2-methyl-.

1. A method for detecting a plant disease at its early stages, comprising steps of:
a. obtaining a plant sample;
b. extracting volatile compounds from said plant sample;
c. optionally, separating said volatile compounds by separation means;
d. optionally, identifying said volatile compounds; and
e. associating said volatile compounds with predetermined volatile reference patterns, wherein, said detecting is executed on a group selected from: inoculated samples compared to non-inoculated samples, different genera of microorganisms causing said plant disease, different species of microorganisms causing said plant disease, different sub-species of microorganisms causing said plant disease, and any combination thereof.

2. The method of claim 1, wherein said plant disease is potato blackleg, soft rot diseases and any combination thereof.

3. The method of claim 1, wherein said plant is potato.

4. The method of claim 1, wherein said plant sample is selected from a group consisting of whole plant, tuber, leaf, root, rhizome, flower, stem, cuttings, discs, macerated samples and any combination thereof.

5. The method of claim 1, wherein said separation means are selected from a group consisting of chromatography and detection or identification means selected from a group consisting of mass spectrometry, spectrophotometry, NMR, near infra-red (NIR), flame ionization detector, nitrogen phosphorus detector, thermal conductivity detector, flame photometric detector, photoionization detector, electrolytic conductivity detector, quadrupole time of flight mass spectrometer, or their detection without separation or without identification by a sensor, a biosensor or an electronic nose or NIR detector and any combination thereof.

6. The method of claim 1, wherein said predetermined volatile reference patterns are generated from plants of the same species as said plant sample, inoculated with microorganisms, analyzed for volatile compounds by said separation and identification means or other means known in the art, or in any other means, and saved as a reference to be compared against said plant sample.

7. The method of claim 1, wherein said volatile compounds are selected from any organic or inorganic chemical produced by said plant.

8. The method of claim 1, wherein said volatile compounds are selected from a group consisting of isoprenoids, terpenoids, alcohols, esters, ketones, alkanes, phenylpropanoids, benzenoids, fatty acids and their derivatives, amino acids derivatives, oxygenated volatile compounds, sulfur-containing compounds, and derivatives thereof and any combination thereof.

9. The method of claim 1, wherein said predetermined volatile reference patterns used to discriminate between said inoculated sample compared to non-inoculated sample are selected from a group comprising: 3,5-Octadien-2-one, E,E-; Carveol; Acetic acid, methyl ester; Benzene, 1,2-dimethoxy; and Dimethyl ether, Valencene; Isopropyl Alcohol; 1,6-Dioxaspiro4.4nonane, 2-ethyl-; Acetoin (3-hydroxy-2-butanone); 2,3-Butanediol; and Octanoic acid ethyl ester and any combination thereof.

10. The method of claim 1, wherein said predetermined volatile reference patterns used to discriminate between said genera of said microorganisms are selected from a group comprising: Hexanal; and 2,4-Heptadienal, E,E, Phenol, 2-methoxy; 2,3-Butanediol, R,R-; 2,4-Nonadienal, E,E-; UK1; 1-Hexanol; Phenol, 4-ethyl-2-methoxy-; and 3-Hepten-1-ol and any combination thereof.

11. The method of claim 1, wherein said predetermined volatile reference patterns used to discriminate between said species of said microorganisms are selected from a group comprising: Thiophene, 2-pentyl, 1-Pentanol, 3-methyl-; 1-Butanol, 3-methyl-, acetate; 1-Butanol, 2-methyl; Valencene, 2-Octen-1-ol, E-; Phenol, 2-methoxy-; Carveol; 1-Butanol, 2-methyl; 1-Hexanol, 1-Pentanol, 3-methyl; 1-Octanol; 2,3-Pentanedione; Cyclopentane, methyl-; 1-Pentanol, 4-methyl-; Phenylethyl Alcohol and any combination thereof.

12. The method of claim 1, wherein said predetermined volatile reference patterns used to discriminate between said subspecies of said microorganisms are selected from a group comprising: 2,3-Octanedione; and 2-Hexenal, 2-methyl, Cyclopentane, methyl; 2-Octenal, E-; 3-Nonen-1-ol, Z-; 1-Octen-3-ol; and 2,4-Nonadienal, E,E-; 1-Butanol, 2-methyl-; 1-Propanol, 2-methyl-; 1-Penten-3-ol; 2-Decenal, Z-; Fenchol; 2-Undecanone; 2,3-Butanediol, R,R; 1-Pentanol, 2-methyl-; 2-Octenal, E-; Cyclopentane, methyl- and any combination thereof.

13. The method of claim 1, wherein said microorganisms are selected from bacteria, fungi, viruses and any combination thereof.

14. The method of claim 13, wherein said bacteria are selected from a group consisting of: Pectobacterium and Dickeya and any combination thereof.
